(19) United States
(12) Patent Application Publication
(10) Pub. No.: US 2006/0246572 A1
(43) Pub. Date: Nov. 2, 2006

(54) AUTOMATIC ELECTROPORATION OPTIMIZATION SYSTEM

(75) Inventors: Charles W. Ragsdale, (US); Julie K. Moore, (US); William C. Heiser, (US); Tony Chiu, (US); Keith Kaste, (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 11/458,039

(22) Filed: Jul. 17, 2006

Related U.S. Application Data
(62) Division of application No. 10/313,893, filed on Dec. 6, 2002.
(60) Provisional application No. 60/337,095, filed on Dec. 6, 2001.

(51) Int. Cl. C12M 1/42

(52) U.S. Cl. 435/285.2

(57) ABSTRACT

Systems, methods and algorithms for automatically performing optimization of an electroporation system. A system according to the present invention typically includes a cuvette holding assembly configured to hold a plurality of electroporation cuvettes, wherein each cuvette includes a first and second electrode, and a shocking chamber configured to hold the cuvette holding assembly, the chamber having a commutator assembly configured to provide an electrical contact to the first electrode of each of the plurality of cuvettes in turn. The system also typically includes a control system communicably coupled to the shocking chamber, wherein the control system controls the commutator to automatically contact the first electrode of each cuvette in an order and to provide a potential across the cuvette electrodes when contact is made.

### CROSS-REFERENCES TO RELATED APPLICATIONS

This application claims the benefit of U.S. Provisional Patent Application Serial No. 60/337,095, titled “AUTOMATIC ELECTROPORATION OPTIMIZATION SYSTEM”, filed Dec. 6, 2001, which is hereby incorporated by reference in its entirety.

### BACKGROUND OF THE INVENTION

The present invention relates generally to electroporation systems and more particularly to systems and methods for automatically optimizing electroporation processes. The present invention also relates to hand-held data transfer apparatus for use with electroporation systems.

A number of parameters cause major and subtle changes in the efficiency of an electroporation process or experiment. Parameters that may cause major changes include the actual organism selected, the preparation of an organism, the gene or other DNA to be inserted, the wave-shape (e.g., square-wave or exponential), the electric field intensity or field strength (determined by actual pulse amplitude and sample cuvette electrode spacing), and the time constant (or pulse length).

Parameters that may cause more subtle changes include slight differences among strains of an organism, slight variations in preparation and preparation components, and subtle variations in electroporation instrument parameters (within the specifications of the actual instrument).

Hence, in order to find the maximum efficiency (typically for future comparative work), an optimization experiment should be run. Such an optimization experiment is generally run manually and typically includes performing electroporation on aliquots of the sample at slightly different settings of the electroporation instrument parameters. Of course, this means that the electroporator must be set to slightly different parameters before each pulse is delivered. Making the necessary changes to the various instrument settings can be time-consuming and is subject to operator error.

Accordingly it is desirable to provide systems and methods for automatically performing optimization of an electroporation system.

### BRIEF SUMMARY OF THE INVENTION

The present invention provides systems, methods and algorithms for automatically performing optimization of an electroporation system. According to one aspect, an operator first selects an auto-optimization mode in the electroporation stystem. A set-up screen is provided on a display which allows selection of various system parameters such as waveform (exponential or square), number of pulses, pulse width, points per experiment, Hi Volts (highest voltage in an experiment), Lo Volts (lowest voltage in an experiment), and Cap (capacitance at which the experiment is run). Other parameters such as sample resistance, resistance in parallel with the sample, and time constant can be added as parameters to control. The optimization algorithm controls the electroporation system to perform one, two or more experiments, each experiment including a series of electroporations. Each experiment allows for plotting a curve using the input parameters from the optimization screen. Two curves allow for the examination of two parameter values and the identification of optimal conditions at the point that the two curves intersect.

A commutator assembly is also provided for use with a cuvette carrousel arrangement. In certain aspects, the cuvette carousel does not rotate, but remains stationary while the commutator assembly rotates. The cuvettes do not turn; rather a commutator finger makes contact to each cuvette.

Hand-held data transfer systems and apparatus are also provided. The hand-held data transfer systems and apparatus of the present invention provide various benefits including: 1) eliminating safety issues by transporting data between a high-voltage electroporation instrument and a desktop computer, using a hand-held unit; 2) incorporating automatic collection of data from an optimization routine using a hand-held unit; 3) providing a simple, inexpensive means to allow automated demonstrations of the product; 4) incorporating a filter to prevent ambient/room light from affecting the infrared transmissions; 5) providing a generalized system that can be incorporated inexpensively in every DNA and Protein instrument; and 6) providing a simple hand-held unit that can be supported over a long period of time.

According to an aspect of the present invention, an automatic electroporation system is provided. The system typically includes a cuvette holding assembly configured to hold a plurality of electroporation cuvettes, wherein each cuvette includes a first and second electrode, and a shocking chamber configured to hold the cuvette holding assembly, the chamber having a commutator assembly configured to provide an electrical contact to the first electrode of each of the plurality of cuvettes in turn. The system also typically includes a control system communicably coupled to the shocking chamber, wherein the control system controls the commutator to automatically contact the first electrode of each cuvette in an order and to provide a potential across the cuvette electrodes when contact is made.

According to another aspect of the present invention, a portable data transfer device is provided for use with an electroporation system. The device typically includes an optical data port configured to send and receive optical data signals to and from an electroporation instrument configured with an optical data port. The device also typically includes a memory for storing data, and a user input module for receiving user input commands. In operation, when a user positions the device proximal the electroporation device, the device transmits stored data to the electroporation instrument responsive to a download command received from the user, and receives and stores data from the electroporation instrument responsive to an upload command received from the user.

According to yet another aspect of the present invention, an electroporation system is provided that typically includes an electroporation unit configured with a data port for sending and receiving data and commands, the electroporation unit including a commutator assembly configured to provide, in turn, an electrical contact to a first electrode of each of a plurality of cuvettes in the unit. The system also typically includes a portable data transfer device configured with a data port for sending and receiving data and commands, and a computer system configured with one or more data ports for sending and receiving data and commands, the computer system executing an optimization module that determines experimental parameters for electroporation experiments in the electroporation unit responsive to user input parameters. The computer system is typically configured to automatically determine a first set of experimental parameters in response to a first set of user input parameters, wherein the user downloads the first set of experimental parameters to the portable data transfer unit using one of the one or more data ports of the computer system. The user transfers the first set of experimental parameters to the electroporation unit using the portable data transfer device, whereby the electroporation unit performs a series of electroporation experiments on the cuvettes responsive to the received experimental parameters.

According to a further aspect of the present invention, a computer readable medium including code for optimizing electroporation experiments is provided. The code typically includes instructions for controlling a processing module to prompt a user to input desired values for one or more parameters, and responsive to the user input values, automatically determining experimental parameters for an electroporation experiment.

According to yet a further aspect of the present invention, a cuvette holding apparatus for holding a plurality of electroporation cuvettes is provided. The apparatus typically includes a carousel shaped body, and a plurality of cuvette receiving elements located in a circular arrangement on the body.

Reference to the remaining portions of the specification, including the drawings claims and Appendices, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

FIGS. 1-11 illustrate various features of cuvette carousels and commutator assemblies according to embodiments of the present invention.

FIG. 12 illustrates features of a hand-held data transfer device according to an embodiment of the present invention.

FIG. 13 illustrates an auto-optimization system for use with an electroporation system according to one embodiment of the present invention.

### DETAILED DESCRIPTION OF THE INVENTION

Auto-Optimization System

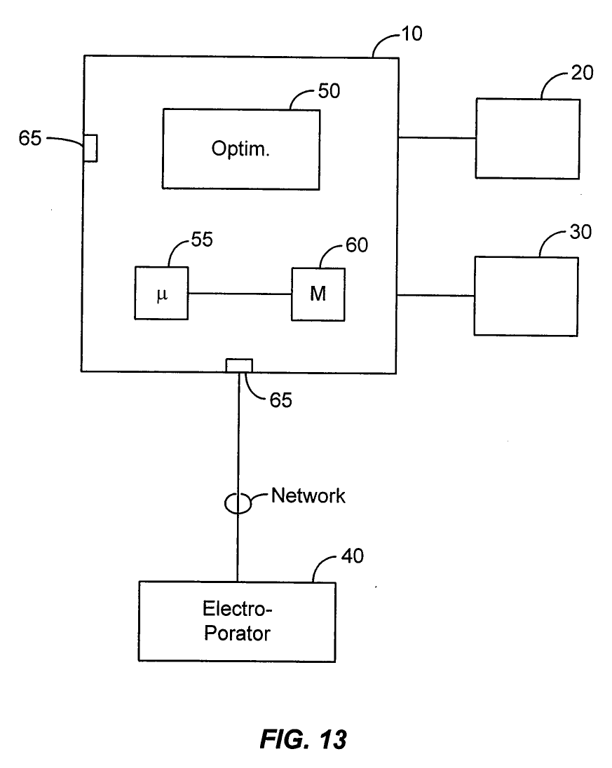

FIG. 13 illustrates an auto-optimization system for use with an electroporation system according to one embodiment of the present invention. The auto-optimization system as shown includes an intelligence module 10 (e.g., computer system, ASIC, microprocessor, etc.) a display 20 (e.g., monitor, LED display, etc.) and a user input device 30 (e.g., mouse, keyboard, buttons, etc.). Intelligence module 10 and the other components may be part of a stand alone or network connected computer system as shown in FIG. 13, or they may be directly attached to or incorporated in an electroporation system or device 40. In preferred aspects, intelligence module 10 includes an optimization software module 50 that executes in a microprocessor module 55. According to one embodiment, application module 50 includes instructions for optimizing and controlling electroporation experiments as described herein based in part on user input parameters. Application 50 is preferably downloaded and stored in a memory module 60 (e.g., hard drive or or other memory such as a local or attached RAM or ROM), although application module 50 can be provided on any software storage medium such as a floppy disk, CD, DVD, etc. In one embodiment, application module 50 includes various software modules for processing data content, such as for communicating data through a data port 65, for rendering displays on display 20, for interfacing with and controlling operations of electroporation system 40 over a network connection, direct connection or indirectly, e.g., via a hand-held device as will be described herein, and for storing data to and retrieving data (e.g., parameters, experiment results, etc.) from memory. It should be understood that computer code for implementing aspects of the present invention can be implemented in a variety of coding languages such as C, C++, Java, Visual Basic, and others, or any scripting language, such as VBScript, JavaScript, Perl or markup languages such as XML. In addition, a variety of languages and protocols can be used in the external and internal storage and transmission of data and commands according to aspects of the present invention.

In one embodiment, an auto-optimization system application operates as follows: (1) Operator selects an auto-optimization mode. (2) Display screen 20, such as a graphical LCD, displays a set-up screen which allows selection of various system parameters such as waveform (exponential or square), number of pulses, pulse width, points per experiment, Hi Volts (highest voltage in an experiment), Lo Volts (lowest voltage in an experiment), and Cap (capacitance at which the experiment is run). Other parameters such as sample resistance, resistance in parallel with the sample, and time constant can be added as parameters to control. Such parameters can be automatically determined, for example, as disclosed in copending U.S. patent application Ser. No. 10/______, (Atty. Docket No. 002558-066710US) filed on even date herewith, claiming priority to U.S. Provisional Patent Application Ser. No. 60/337,103, filed Dec. 6, 2001, both titled “RESISTANCE CIRCUIT STABILIZATION AND PULSE DURATION CONTROL SYSTEMS FOR ELECTROPORATION INSTRUMENTS”, the contents of which are both hereby incorporated by reference in their entirety. (3) An example of an optimization screen is as follows:

| | Exp1 | Exp2 |
|---|---|---|
| Waveform | — | — |
| No. Pulses | — | — |
| PIs Width | — | — |
| Pis Interval | — | — |
| Pts/Exp | — | — |
| Hi Volts | — | — |
| Lo Volts | — | — |
| Cap | — | — |

The optimization algorithm 50 controls the electroporation system 40 to perform one, two or more experiments, each experiment including a series of one or more electroporations. These experiments allow plotting curves using the input parameters from the optimization screen. Processing two curves allows for the examination of two parameter values and the identification of optimal conditions at the point that the two curves intersect. For example, voltage is often a sensitive parameter. One usually wants to find the voltage at which the efficiency of an electroporation experiment is maximum. However, some other parameter (such as capacitance) may be of interest. Of course, the goal is to find the set of parameters which gives the best results. Those parameters would usually be used for future experiments for the particular cells and vectors. Even if the user is repeating the published protocol of some other researcher, it is likely necessary to run the optimization experiment due to the previously stated potential variables.

As an example, one could select an exponential waveform (e.g., can toggle between exponential and square; however, exponential is preferably the default). The system automatically defaults to exponential for experiment 2 (subject to change if desired). The number of pulses is automatically defaulted to one (for both experiments), but can be changed as desired. One pulse (per sample) is the typical set-up desired. Pulse width is not applicable for an exponential waveform, so one next enters the number of points per experiment, e.g., 10-25 or more. Assume that 25 is entered for the number of pulses (same number automatically entered for experiment 2, subject to change by the user). Next, assume that the user enters 1.8 KV for Hi Volts and 1.2 KV for Lo Volts. Since, typically, one wishes to use the same voltage range for experiment 2, the entered values preferably default to experiment 2 (subject to change by the user). Assume that one is also interested in the effect of capacitance on optimization; one could then enter a capacitance, e.g., 25mfd, for experiment 1 and a different capacitance, e.g., 50mfd, for experiment 2.

After the relatively quick entry of just the five indicated parameters, the optimization algorithm automatically sets-up the electroporation system to sequentially deliver a series of pulses corresponding to the voltage range entered by the user, in this example 1.2 KV to 1.8 KV. The system automatically determines the voltage intervals for the given range based on the number of pulses selected, and controls the electroporation system to deliver pulses to the samples/cuvettes as follows (for the present example the system set precision is 0.01 KV; i.e., the system rounds-off to the next 0.01 KV voltage increment):
25mfd capacitance
(a) 1.2 KV
(b) 1.23 KV
(c) 1.25 KV
(d) 1.28 KV
(e) 1.3 KV
(f) 1.33 KV
(g) 1.35 KV
(h) 1.38 KV
(i) 1.4 KV
(j) 1.43 KV
(k) 1.45 KV
(l) 1.48 KV
(m) 1.5KV
(n) 1.53 KV
(o) 1.55 KV
(p) 1.58 KV
(q) 1.6 KV
(r) 1.63 KV
(s) 1.65 KV
(t) 1.68 KV
(u) 1.7 KV
(v) 1.73 KV
(w) 1.75 KV
(x) 1.78 KV
(y) 1.8 KV
50mfd capacitance
(a) 1.2 KV
(b) 1.23 KV
(c) 1.25 KV
(d) 1.28 KV
(e) 1.3 KV
(f) 1.33 KV
(g) 1.35 KV
(h) 1.38 KV
(i) 1.4 KV
(j) 1.43 KV
(k) 1.45 KV
(l) 1.48 KV
(m) 1.5 KV
(n) 1.53 KV
(o) 1.55 KV
(p) 1.58 KV
(q) 1.6 KV
(r) 1.63 KV
(s) 1.65 KV
(t) 1.68 KV
(u) 1.7 KV
(v) 1.73 KV
(w) 1.75 KV
(x) 1.78 KV
(y) 1.8 KV

Hence, the mere entry of five parameters (in this example) automatically sets-up 50 data points. However, one typically needs to press a pulse button, remove the old cuvette, and load a new cuvette for each pulse condition. Thus, it would be even more desirable to eliminate such manual pulsing and cuvette replacement to save further time and increase operator efficiency.

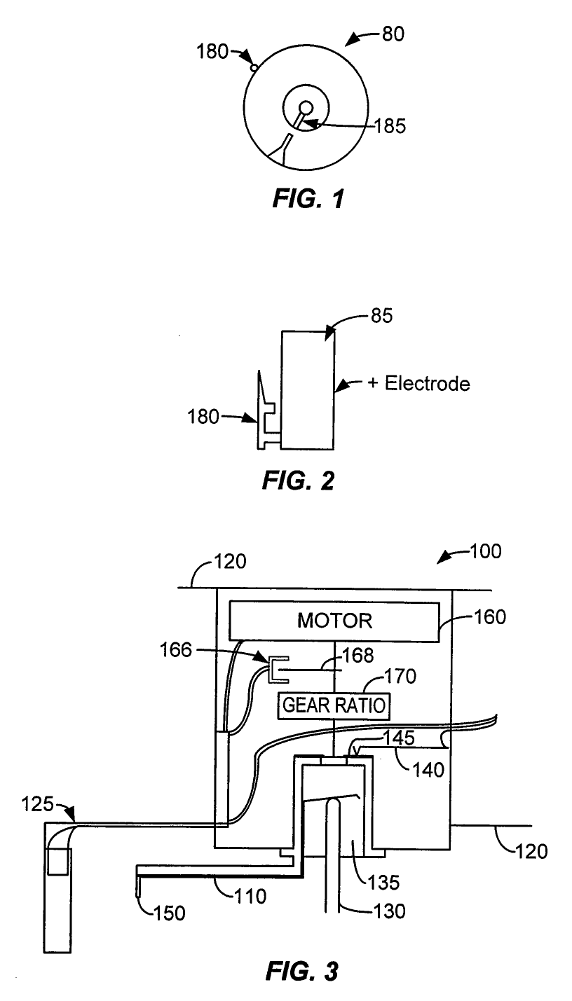

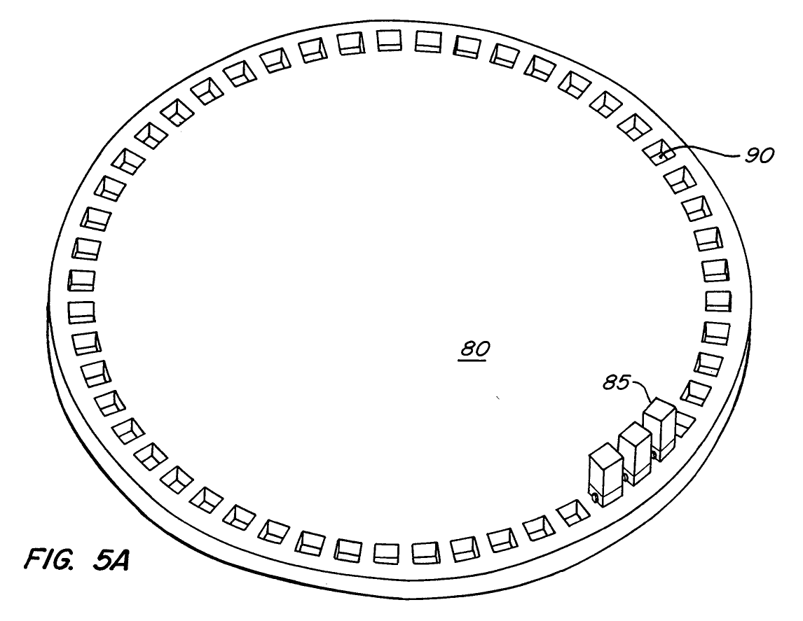

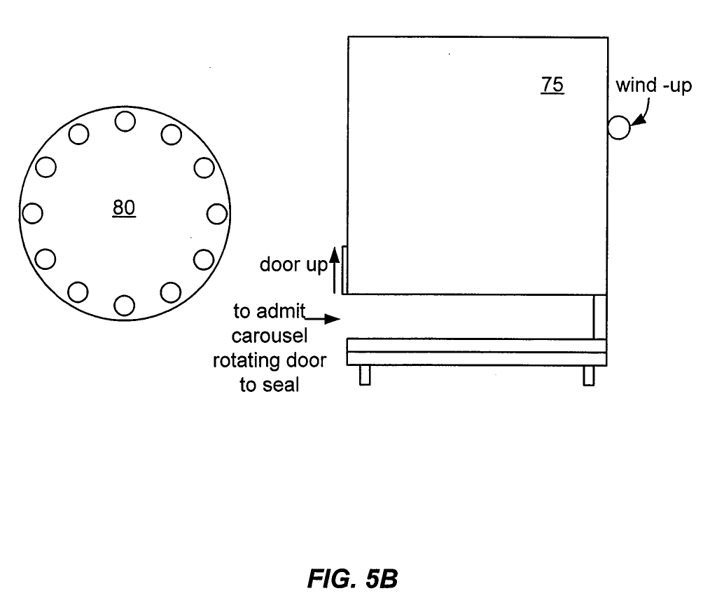

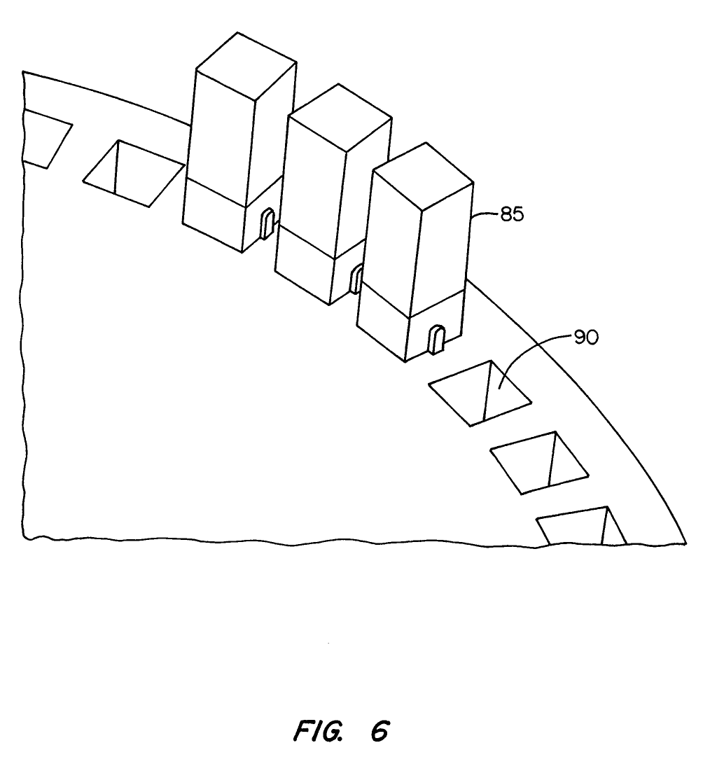

According to another embodiment of the present invention, an auto-advance shocking chamber is provided. FIGS. 1, 5a, 5b and 6 illustrate aspects of a shocking chamber 75 and cuvette carousel according to one embodiment of the present invention. Such an auto-advance shocking chamber 75 is preferably configured and shaped to receive a carousel 80, but could take other shapes. Carousel 80, and therefore chamber 75, is preferably configured to hold multiple, e.g., up to fifty or more, cuvettes 85 in cuvette holding spaces 90. The chamber 75 also preferably includes a means to keep the cuvettes cold, e.g., using blue ice. The chamber 75, in one embodiment, includes a motor such as a solenoid and ratchet and pawl mechanism to advance the carousel, cuvette by cuvette, to make contact with the shocking electrodes for each cuvette. The solenoid receives a power pulse from the electroporation system as the time for cuvette advancement arrives. If the auto-advance shocking chamber is implemented, pressing a pulse button, for example, starts the automatic delivery of each pulse, advancing to the next cuvette in time for the next pulse. Hence, in this embodiment, all that is required for set-up is entering the (five) parameters, and pressing the pulse button. This is a clear time saver and prevents potential set-up errors while performing electroporation. In one embodiment, the pulse conditions at each new pulse are displayed before the pulse is delivered. Likewise, at the end of the set of pulses, the operator is given the choice of repeating the set of pulses, any individual pulse(s) or ending the pulse delivery session.

Also, in some cases, many cuvettes may not be able to be electroporated in sequence because some chemical addition must be made to a sample within a certain time period. Thus, in one embodiment, the operator loads as many cuvettes as practical, and the auto advance occurs. However, when the first empty cuvette space 90 in the carousel 80 is reached, the system stops. For example, the system in certain aspects measures sample resistance to determine whether a cuvette is present. The first cuvettes are offloaded, chemicals added, and the next batch loaded on the carousel. The auto routine is configured to pick up from where it left off when the pulse button is pressed.

Such an algorithm according to the present invention provides a huge savings in keystrokes and potentially prevents entry errors which may occur if the system settings would be changed before each pulse. Also, the delay required for making each setting is eliminated thus preventing potential damage to sensitive cells. Finally, the actual achieved parameters (e.g., voltage and time constant) for each pulse are stored and the full results of an optimization experiment are output to a memory unit, printer or a computer in one embodiment. This results in a further time savings and decrease in potential transcription errors.

When performing electroporation experiments, many individual data points (and thus cuvettes) may be used. According to certain aspects:
(1) For optimization experiments, the user selects cell type and media; stored electroprotocols can provide a starting point.
(2) Cuvette already in carousel would be on ice; carousel placed in shocking chamber and door closed; gel pack keeps cold.
(3) Spring-driven motor provides power.
(4) Shocking chamber main contains small release solenoid to allow movement position to position.
(5) System accepts input of how many cuvettes; optimizes on volts or volts+RC depending on number of cuvettes. Download data from IR port.
(6) System chooses setting around nominal; start pulses, automatically finishes; alarms.

For Std. Experiments:
(1) Set-up exp protocol at office computer; download to programmer.
(2) Programmer uploads data to electroporation system.
(3) Insert carousel.
(4) Start pulses.
(5) Completes automatically and alarms.
(6) Download data to programmer through IR.

Different carousels can be configured to accept different cuvette types.

In certain aspects, the optimized electroporation experiments need not be carried out fully automatically. For example, in a standard electroporation instrument configured to hold a single cuvette, a user may be prompted to place a different cuvette into the system for each experiment or pulse, however, the algorithm will control the system to provide the desired pulse to the inserted cuvette. Additionally, the system or algorithm may be configured to prompt a user to place each cuvette in-turn into a shocking chamber.

Cuvette Carousel and Commutator Assembly

In order to implement the automated portion of the optimization system, according to one embodiment a cuvette holder is provided that includes the following features:

(1) Interlocked so that users cannot contact high voltage

(2) Accepts multiple, e.g., up to 50 or more, cuvettes

(3) Commutates to each cuvette in-turn

(4) Provides blue ice pockets for cuvette cooling

(5) Uses a relatively small motor (e.g., 1 A 12V max).

In preferred aspects, the cuvette holder is shaped like a round carousel as discussed above, however, it should be appreciated that alternate geometries may be implemented. The electroporation (transfection) system of the present invention includes hardware and software to interface to the cuvette carousel. By these means, a user can initiate an optimization experiment (as discussed above) and pulse the cuvettes automatically. The user accesses the optimization program, enter the parameters requested, inserts the cuvettes in the carousel, replaces or closes the lid, and presses a pulse or start button, or otherwise initiates an experiment. The system pulses each cuvette at a slightly different setting and advances the cuvette to the next position. This is a tremendous time saver and eliminates the error inherent in setting the electroporator for each pulse. Data is collected at each pulse for retrieval and/or storage at the end of the optimization experiment.

According to one embodiment of the present invention as shown in FIGS. 1-6, a commutator assembly 100 (FIG. 3) is provided for use with a cuvette carrousel arrangement (FIGS. 1, 5-6). In preferred aspects, the cuvette carousel does not rotate, but remains stationary while the commutator assembly 100 rotates. The cuvettes do not turn; rather a commutator finger 110 makes contact to each cuvette. The finger 110 is a light assembly thereby reducing the torque required for the system. In addition, since the cuvettes do not turn, the assembly does not need to be kept level (e.g., the assembly can be placed on a pile of ice to keep cold).

In one embodiment, the system includes a fail-safe interlock to prevent operator contact with high voltage. As shown in FIG. 3, the commutator assembly is preferably integrated with the lid 120. When the lid is removed, the negative contact 125 (e.g., a long-throw connector; may include two for balance and mechanical latching) disconnects, and an interlock finger 130 pulls out of the hole 135 in the commutator assembly 100. When the interlock finger 130 is pulled through the hole 135 , an internal contact 140 separates from a slip-ring assembly 145 (thereby producing the needed separation, e.g., 0.6 inch or more). This disconnects the positive electrode 150 on the tip of the commutator finger 110. Hence, both sides of the high voltage are disconnected.

In one embodiment, the system finds home by moving the commutator finger to a shorted cuvette position. The motor steps the commutator finger until a shorting contact is found. This puts the commutator assembly in position for removal. Shorting allows knowledge by the system that the motor (e.g., stepper) has moved the commutator finger to the right position. Having low resistance as the indicator is more reliable than high resistance. Cuvette samples should have a low resistance, e.g., a minimum of about 20 ohm. However, high resistance samples would be beyond the ability to measure opens. Having the commutator in the correct position allows stepping to the approximate center of each cuvette electrode with reasonable accuracy.

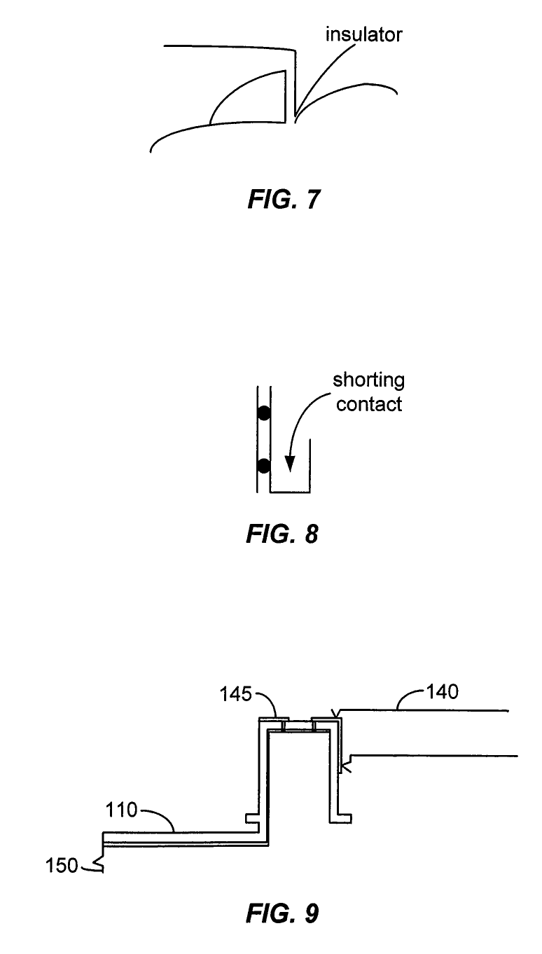

One of the problems with the aforementioned shorting method is that the distance between cuvette positions on a small commutator is small. One could place an insulator as shown in FIG. 7, but it would be necessary to have good accuracy. In a typical arrangement according to the present invention, for 50 cuvettes, each cuvette position is about 0.050″ from the next. Hence, one cuvette position would be sacrificed (still allows 50 cuvettes) and a shorting contact installed as shown in FIG. 8. This gives a greater circumference and removes shorting accuracy problems.

In addition, a run mode is provided in software to run the cuvette commutator finger 110 forward or back should it inadvertently be pushed out of position. Basically, the commutator finger is moved to just “kiss” the negative connector assembly; this negative connector is preferably designed so as to “use-up” only one cuvette position.

By finding the correct position over the circumference of the carousel, the dimensional accuracy is reduced from that required if the homing was performed within the commutator assembly. The resistance circuit of the electroporation system is capable of reasonably accurate readings in the 5-10 ohm range. If finding an open were the method employed, the resistance circuit would not have a sufficient range, since the circuit could not tell the difference between an open and that of a high-resistance cuvette.

In one embodiment, the commutator assembly 100 includes circuitry that allows the motor 160 to be run continuously or in a step mode. When providing a 98% duty cycle pulse train to the motor (by means of a 12V supply), the motor moves essentially continuously. This mode is used to find home (shorted cuvette). Lifting the line (e.g., 12V) for some period (e.g., 0.75 sec) and then dropping the line causes the motor to run until a vane 168 interrupts the slotted limit switch 166. This stops the motor. Placing the limit switch before the gear train 170 reduces the angular accuracy required. The gear train 170 is selected to provide adequate torque, and is used with the positioning of the cuvette centers such that one rotation of the motor is one advance to the next cuvette.

As shown in FIGS. 1 and 2, in one embodiment, a single continuous negative contact 180 connects all cuvettes (and shorts all outside contacts together). This simplifies the system. Blue ice packs or other cooling means are preferably positioned in the chamber, allowing samples to be kept cold. In certain aspects, the commutator finger assembly is constructed so that a ledge acts as a bearing surface to the bottom of the commutator assembly. A ball bearing or other type of bearing arrangement can be used. In one embodiment, a slot 185 is provided to assure that the lid cannot be removed until the system finds home.

In operation, the operator lifts the commutator assembly, which was first stepped to the shorting position by the system. If not in this position, the commutator assembly preferably cannot be removed. When the commutator assembly is in place, the operator cannot touch the cuvettes. The system knows it is in the shorting position by measuring resistance. The system runs the motor until a low-resistance is detected. Removing the commutator assembly exposes the cuvette positions. The operator inserts cuvettes and makes sure that blue ice is in pockets in the assembly. When the commutator assembly is removed, the negative contact (made through long-throw connector into the commutator assembly) is broken. The operator cannot touch the negative contact in the commutator assembly. Also, the center finger no longer presses on the internal+contact so that contact with the+slip-ring assembly is not made. It is possible to eliminate this assembly since the commutator must be in the short position when removing the assembly. If this is the case, the center portion of the commutator assembly can be designed as shown in FIG. 9.

Hence, the cuvettes are put in place, and the commutator assembly is replaced. The optimizer routine is started, and the commutator finger steps to each cuvette in-turn. The system uses cuvette resistance change as a guide.

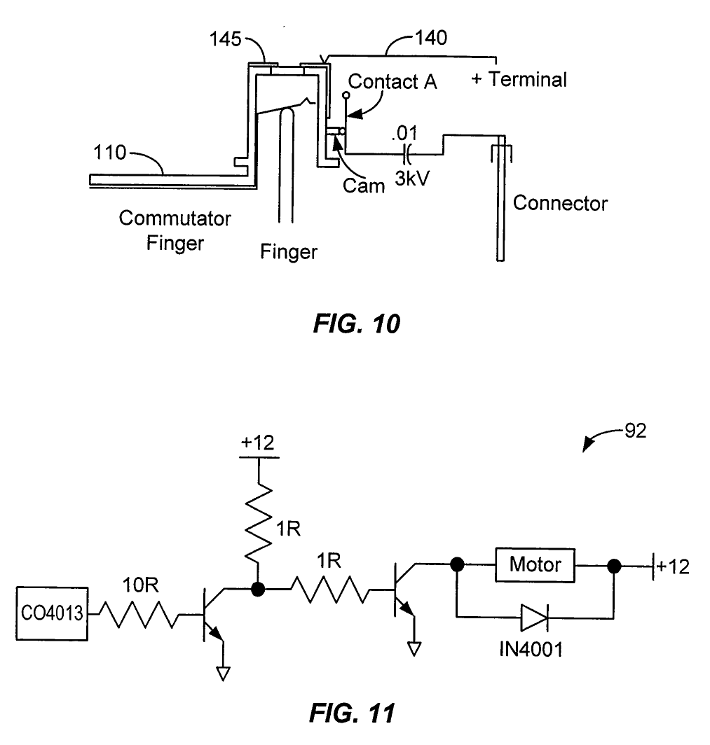

In some designs, the electronics of the system may not be adaptable to a stepper motor; there may be no lines which conveniently allow the measurement of current. Otherwise, one could switch a resistor in parallel to change driver current, tying the switching of the resistor to being in the right position. With reference to FIG. 10 an alternate design is as follows:
(1) A stop is positioned inside the commutator assembly which stops when the assembly is lined with home.
(2) The shorting bar is eliminated.
(3) Contact A places a capacitance (e.g., approximately 0.01mfd capacitance) across the sample in-between the cuvettes, and lifts at the point the motor should stop.

In this example, since the reactance of the capacitance is about 600 ohms at 25 KHz, this gives a noticeable change in sample resistance. If sample resistance is about 20 ohms, the sample resistance goes to about 600 ohms and then changes (drops). When the microprocessor sees the change either goes up or down, the motor is stopped. The cam can be made of a large ring to increase the accuracy.

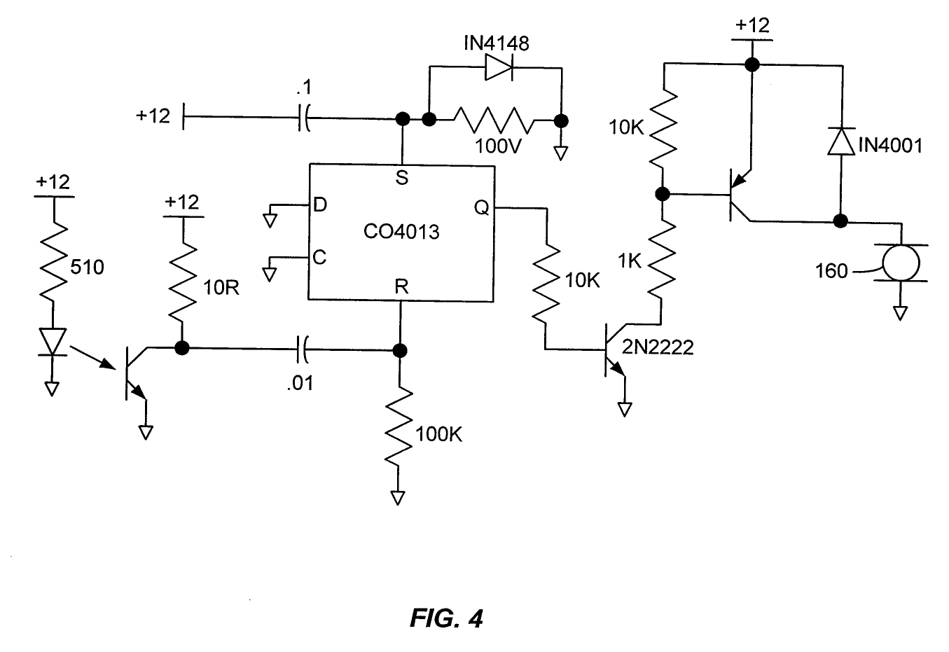

There may be some problems with the previous design. For example, the system may only produce an on-off 12V drive. Hence, a motor could only go in one direction. Hence, the commutator finger should be capable of 360 degree operation. It should be possible to run a motor essentially continuously in order to find home (shorting cuvette position). Thus, the cuvette carousel preferably includes circuitry as shown in FIG. 4.

The system works as follows
(1) Slotted limit switch picks-up one revolution of motor; multiple slots could be used as necessary.
(2) A gear train increases torque and provides proper positioning for each cuvette.
(3) When 12V is applied, the flip-flop is set, turning on the motor. The motor runs until the next position at which time the action of the slotted limit switch resets the flip-flop.
(4) If essentially continuous running of the flip-flop (motor) is desired, the system switches the 12V supply, e.g., 100 microseconds off and 5 msec on. Since the set line has a 10 msec delay, the set line will be held high for essentially 5 msec and low for 100 microseconds. The 1N4148 resets time. The 100 ohm resistor resets the motor.
(5) Another way to keep the 12V line from rising during motor back EMF is to include a circuit as shown in FIG. 11.

One software algorithm is as follows:
(1) Allow movement of motor by pressing front-panel key to align armature commutator finger with slot if it becomes misaligned.
(2) Verify that shorting occurs when the lid is placed; if not, run motor by pulsing 12V until short is detected.
(3) For each cuvette position, raise 12V for 1000 msec; commutator will move to next position and stop.
(4) Operator has entered no. of cuvettes in each block. 12V is briefly dropped and raised 1000 msec to go to next cuvette position. This continues until block no. is reached.
(5) The commutator finger is then made to find home and lid removed.

Optical Data Transfer System

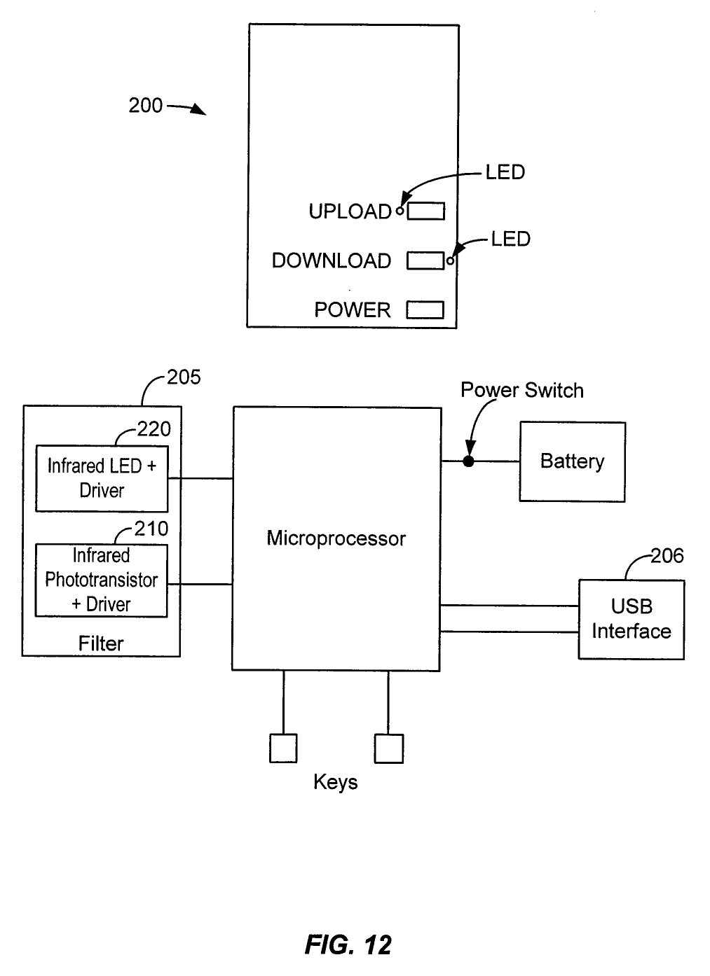

The present invention also provides in one embodiment, an optical data system for use with an electroporation system. The optical data system of the present invention includes a hand-held input/output device 200 as shown in FIG. 12, which interfaces with a data port (e.g., optical port, USB, etc.) in the electroporation system. The hand-held input device200 is preferably designed to be battery-operated but also allow use while connected to an AC adaptor/battery charger. The hand-held device 200 preferably includes one or more of an optical (e.g., IR,) port 205 as well as RS-232, parallel, and a USB ports 206 (can be one or all; e.g., standardize on USB) for connection to a printer or for connection to a user's computer.

The optical data system provides the following functions:
(1) Input of set-up parameters at a desktop, transport of set-up parameters by means of the hand-held unit, and upload of the set-up parameters to an electroporation system.
(2) Download of electroporation data to the hand-held unit for transport to a desktop computer system for analysis, display, and print-out.
(3) Print-out of data from the hand-held unit.
(4) Upload of new protocols to the electroporation system.
(5) Upload of a demo routine to the electroporation system since the hand-held unit has access to all key presses. For example, such a demo routine could be used with a PowerPoint presentation on a laptop to demonstrate the electroporation system with actual key presses by a less-trained person using an automated program.
(6) Upload new software and data to the electroporation system.

The hand-held unit design allows download of set-up parameters directly from a user desk computer. The hand-held unit design also allows upload of the same set-up parameters to the electroporation system. After either manual or automated delivery of the pulses defined by the set-up parameters, the electroporation system downloads the results of each pulse (e.g., up to 100 for five sets of replicates) to the hand-held unit through a data port, e.g., optical data port, USB port, etc. The hand-held unit is also capable of uploading the latter indicated data to a computer system through one of the hand-held unit's ports. Further, the hand-held unit is capable of interfacing to a standard printer through one of the hand-held unit's ports.

The optical port of the hand-held unit preferably includes a phototransistor 210 and an infrared LED 220. The optical port of the electroporation system also preferably includes the same components. The microprocessor in each unit controls the LEDs, e.g., to turn-off and turn-on the infrared LED. The phototransistor is used by the system microprocessors to receive and decode infrared signals. A filter to reduce the ambient/room light is preferably incorporated in the system. This ambient light can cause significant 120 Hz pickup (e.g., from fluorescent lamps). In certain aspects, the hand-held unit contains a CMOS processor and is battery operated. Isolating the external components using an optical data link and utilizing battery operation eliminates safety issues when attaching external circuitry to a high-voltage instrument. In one embodiment as shown in FIG. 12, the hand-held unit preferably does not include a display (but may easily be modified to include a display if desired). As shown, three keys (on/off, upload, and download) allow simple interface to the system. In this embodiment, the handheld unit acts mainly as a bit bucket for storing data.

An example use of the optical data transfer system is as follows.

The user, at a desk computer, designs an experiment, determines the experimental protocol, and selects the shocking parameters for each part of the experiment to be performed with the electroporation system. Up to 100 or more shocking points are allowed. However, a set of shocking points may be repeated as many times as desired. In certain aspects, the software that resides on the user computer utilizes a spreadsheet (e.g., Excel) and contains drivers to interface to the hand-held unit through an RS-232, parallel port, optical port, USB port, etc. In this manner, the user can easily download the set of shocking points to the hand-held unit.

The user carries the hand-held unit to the electroporation unit, selects the data input screen, places the electroporation system in the shocking point upload mode, and activates the shocking point output feature of the hand-held unit. The shocking points are automatically uploaded through the selected connection port to the electroporation system. In the case of an optical connection, the hand-held unit need only be held within a short distance, e.g., 1-10 inches to several feet, of the optical port of the electroporation system. The user is able to scroll through each of the set-up screens for each of the shocking points for review or change. The user may home to the first point screen, insert a cuvette, and pulse. This can be repeated for each of the shocking points. If the auto shocking chamber is available, the user need only to load the carousel of the auto shocking chamber with as many cuvettes as desired, and press start. Pulses will automatically be delivered by the electroporation system based on the uploaded parameters from the hand-held unit.

If only some of the cuvettes of the data set can be loaded due to the requirement of intervention needs or other reasons, the unit will stop when it finds an open slot (by resistance measurement). One can remove the cuvettes already pulsed, perform the intervention, load the next set, and continue the set of data points until completion. Replicates can be performed by merely repeating the set of shocking points.

Following each set of 100 points (or less or more), the user can access the download screen and output/transfer data to the hand-held unit. This can be performed up to five or more times; hence, the hand-held unit preferably includes memory space for 500 or more sets of shocking parameters. The user carries the hand-held unit to the desk computer, and uploads the data to the system application program in the desk computer. Hence, all of the set-up points and results would be available at the desk computer for printing or other use. Finally, the hand-held unit allows printing directly to a printer.

The firmware of the electroporation system allows interface to the hand-held unit as previously indicated. In preferred aspects, the electroporation system firmware contains a protocol that allows a user to adjust any parameter of the electroporation system and effectively press any electroporation system key using the hand-held unit. This allows the hand-held unit to be used for canned demo programs with an actual electroporation system. In addition, it is possible to load new firmware into the electroporation system by means of the hand-held unit, and the electroporation system is designed to contain protocols to effect the process. Finally, by the latter process, it is possible to re-load any or all of the canned protocols stored within the electroporation system. This allows changes to protocols as necessary, e.g., for future changes in the field of gene transfer.

The optical data system of the present invention can also be applied to a wide range of DNA and Protein instrumentation products. Advantageous features of such a generalized system include the following:
(1) Each instrument preferably includes an infrared port and software that allows upload of set-up parameters/protocols, download of data, upload of a demo routine (has access to all key presses and display info available to an instrument operator), upload of software upgrades, and utilization of troubleshooting algorithms.
(2) A hand-held unit may be made available for purchase at a later date to add the features of the system(s).
(3) Software is available for installation on a user's desktop computer system, e.g., PC. This software interfaces with standard software packages (such as Excel) and allows the user to create new set-up parameters and protocols. The PC downloads the data to the hand-held unit for transport of the data to the vicinity of the product. The user puts the product in the upload mode and pushes upload on the hand-held unit (an LED indicates that upload is in process). New set-up parameters and protocols would be uploaded.
(4) As the product produces data, the data is available for download to the hand-held unit. The user puts the product in the download mode, presses download on the hand-held unit, and data is downloaded (e.g., an LED indicates that download is in process). The user then carries the data in the hand-held unit to the PC for upload, manipulation, display, and printing.
(5) The hand-held unit has a printer port such that data can be printed-out directly from the hand-held unit.
(6) The hand-held unit has access to every possible keypress and display (or LED or LCD) parameter. Hence, a demo routine can be run using the hand-held unit. This feature could be used if, for example, a sales person was not available who could perform an adequate demo of the product. Customers typically want to see the actual product demonstrated (not just a PowerPoint demo). One could start a PowerPoint demo simultaneously with the hand-held unit demo. The product would then go through the motions of actual use as the PowerPoint demo proceeds.
(7) Special troubleshooting algorithms can be utilized by means of the hand-held unit.
(8) New software/firmware can be uploaded into the product to facilitate field upgrades.

While the invention has been described by way of example and in terms of the specific embodiments, it is to be understood that the invention is not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements.

1-14. (canceled)

15. A computer readable medium including code for optimizing electroporation experiments, the code including instructions for controlling a processing module to:
prompt a user to input desired values for one or more parameters;
responsive to the user input values, automatically determining experimental parameters for an electroporation experiment.

16. The computer readable medium of claim 15, wherein the code further includes instructions to:
control an electroporation instrument to perform a series of one or more electroporation experiments according to the determined experimental parameters.

17. The computer readable medium of claim 15, wherein the electroporation instrument includes a plurality of cuvettes, and wherein the instructions to control include instructions to:
automatically apply a series of electroporation pulses of differing values to the cuvettes in an order.

18-20. (canceled)
